(19) United States
(12) Patent Application Publication
(10) Pub. No.: US 2003/0065291 A1
(43) Pub. Date: Apr. 3, 2003

(54) Syringe having flexible barrier between syringe body and plunger

(76) Inventor: Richard F. Corrigan JR., Wheaton, IL (US)

Correspondence Address: HOVEY WILLIAMS TIMMONS & COLLINS, 2405 GRAND BLVD., SUITE 400, KANSAS CITY, MO, 64108

(21) Appl. No.: 09/682,643

(22) Filed: Oct. 1, 2001

(51) Int. Cl. A61M 5/32

(52) U.S. Cl. 604/199

(57) ABSTRACT

A syringe having a flexible barrier coupled to the syringe body and the plunger and operable to prevent contaminants from entering into an injection fluid disposed in the syringe body through the opening in the rear portion of the syringe body.

### BACKGROUND OF INVENTION

1. Field of the Invention

The present invention relates generally to syringes. The present invention further concerns an injection syringe having a flexible barrier between the syringe body and the plunger for preventing contamination of an injection fluid disposed within the syringe.

2. Description of the Prior Art

Injection syringes for injecting various fluids into the arterial, veinous, muscular, and cavity structures of a patient are widely known and used. Such syringes typically include a tubular body having a wide rearward opening for receiving a plunger and a narrow forward fluid passageway for drawing injection fluid into the syringe and expelling injection fluid out of the syringe.

To fill a conventional syringe, the plunger is initially advanced forward as far as possible towards the fluid passageway. The fluid passageway is then coupled to an injection fluid source via a needle or other means. The plunger is then retracted rearward to create a vacuum within the syringe and draw the injection fluid into the body of the syringe through the fluid passageway. Once filled to the desired volume of injection fluid, the plunger can be advanced within the body to thereby force the injection fluid out through the fluid passageway and into the patient.

A significant disadvantage of conventional injection syringes is that when the plunger is advanced towards the front of the body during filling, the inner surface of the syringe wall which defines a fluid-holding chamber in the syringe is exposed to contaminants present in the external environment, including contaminants on the hands of the operator. When the plunger is thereafter retracted to draw the injection fluid into the fluid chamber, contaminants residing on the inner surface of the wall can enter the fluid chamber, thereby contaminating the injection fluid.

### SUMMARY OF INVENTION

In accordance with one embodiment of the present invention, a syringe comprising an elongated shaft, a plunger head, and a barrier is provided. The elongated shaft presents first and second ends. The plunger head is coupled to the first end of the elongated shaft. The barrier is sealingly coupled to the shaft at an attachment location generally centrally positioned between the first and second ends of the shaft.

In accordance with another embodiment of the present invention, a syringe comprising a body, a plunger head, an elongated shaft, and a barrier is provided. The body includes a front portion, a rear portion, and an elongated barrel extending between the front and rear portions. The plunger head is received in the barrel and shiftable relative to the barrel between an advanced position in which the plunger head is located proximate the front portion and a retracted position in which the plunger head is located proximate the rear portion. The elongated shaft is coupled to the plunger head and extends out of the body at the rear portion. The barrier is sealingly coupled to the body and the shaft. The barrier is coupled to the shaft at an attachment location which is positioned generally centrally along the barrel when the plunger head is in the advanced position.

In accordance with a further embodiment of the present invention, a syringe for preventing entry of contaminates from an external environment into an injection fluid disposed in the syringe is provided. The syringe comprises a body, a plunger head, an elongated shaft, and a barrier. The body includes a front portion, a rear portion, and an elongated barrel extending between the front and rear portions. The rear portion presents an opening and the barrel presents an inner surface. The barrel consists of a front zone, a middle zone, and a rear zone, each having equal lengths. The plunger head at least substantially sealingly contacts the inner surface and is shiftable relative to the body between an advanced position wherein the plunger head contacts the front portion and a retracted position wherein the plunger head contacts the rear portion. The inner surface and plunger head cooperate to at least partly define an injection fluid chamber of variable volume. The elongated shaft is coupled to the plunger head and at least partly extends through the opening in the rear portion. The barrier is sealingly coupled to the rear portion and the elongated shaft. The barrier is coupled to the shaft at an attachment location which is positioned in the middle zone when the plunger head is in the advanced position.

In accordance with a further embodiment of the present invention, a method of assembling a syringe is provided. The method includes the step of sealingly coupling a flexible barrier to an elongated shaft of a plunger at an attachment location generally centrally positioned on the elongated shaft.

The present invention provides a more sanitary syringe wherein the injection fluid is protected from contaminants entering the syringe through the opening at the rear of the syringe body.

Other aspects and advantages of the present invention will be apparent from the following detailed description of the preferred embodiment and the accompanying drawing figures.

### BRIEF DESCRIPTION OF DRAWINGS

A preferred embodiment of the present invention is described in detail below with reference to the attached drawing figures, wherein:

FIG. 1 is a perspective view showing the front portion of a syringe constructed in accordance with the principles of the present invention;

FIG. 2 is a perspective view of the rear portion of the syringe;

FIG. 3 is a cross-sectional view of the syringe with the plunger being in the retracted position; and

FIG. 4 is a cross-sectional view of the syringe with the plunger being in the advanced position.

### DETAILED DESCRIPTION

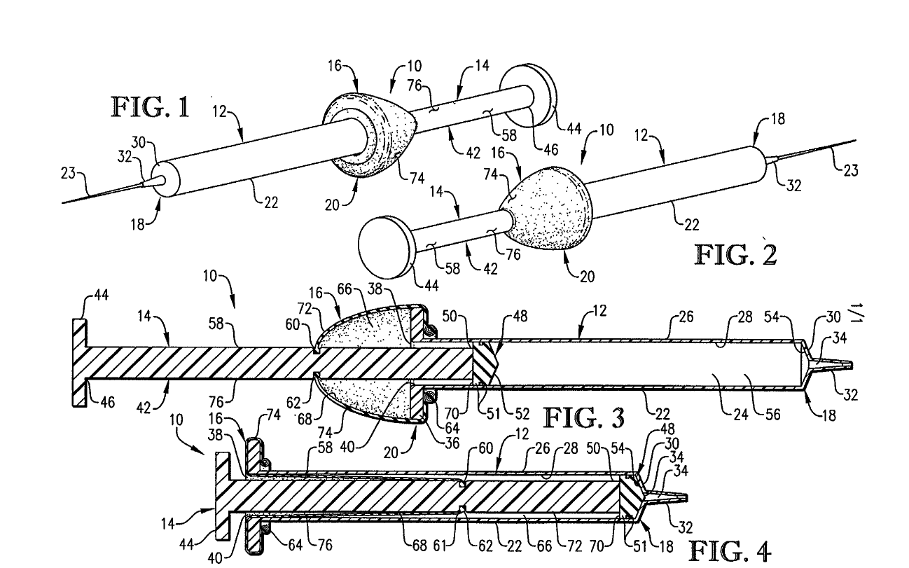

Turning initially to FIGS. 1 and 2, the syringe 10 selected for illustration generally comprises a body 12, a plunger 14, and a barrier 16. Barrier 16 is sealingly coupled to and extends between body 12 and plunger 14. Body 12 generally comprises a front portion 18, a rear portion 20, and an elongated barrel 22 extending between front portion 18 and rear portion 20. Front portion 18 is preferably configured to be releasably coupled to a device for facilitating injection of a fluid into a patient such as, for example, a needle 23.

Referring now to FIGS. 3 and 4, barrel 22 of body 12 defines a main chamber 24 into which plunger 14 may be inserted and shifted relative thereto. Barrel 22 preferably comprises a wall 26 presenting a generally cylindrical inner surface 28 which at least partly defines main chamber 24.

Front portion 18 of body 12 includes a transition wall 30 and a nozzle 32. Transition wall 30 is generally frustoconical in shape with its wide end being coupled to the front of barrel 22 and its narrow end be coupled to the rear of nozzle 32. Nozzle 32 is preferably frustoconical in shape and is adapted to be coupled to a standard needle or other injection-facilitating device. Nozzle 32 defines a fluid passageway 34 which is in fluid communication with main chamber 24.

Rear portion 20 of body 12 includes a flange 36 and a rear opening 38. Flange 36 is generally annular cylindrical in shape and protrudes from the rear of barrel 22. Flange 36 projects from barrel 22 in a direction which is transverse to the direction of elongation of barrel 22. Flange 36 projects generally outwardly from an inside surface 40 of flange 36 which defines opening 38. Inside surface 40 of flange 36 has generally the same diameter as inner surface 28 of barrel 22. Rear opening 38 is sized to allow plunger 14 to be inserted therethrough and into main chamber 24. In an alternative embodiment, not shown, flange 36 projects from barrel 22 at a location spaced from rear opening 38. In such an embodiment, opening 38 is defined by inner surface 28 of wall 26 at the rearward end of barrel 22.

Plunger 14 generally includes an elongated shaft 42, a disk 44 coupled to a proximal end 46 of shaft 42, and a plunger head 48 coupled to a distal end 50 of shaft 42.

Plunger head 48 of plunger 14 is configured to be slidably received in main chamber 24 of body 12. The outer perimeter of plunger head 48 preferably includes annular sealing portions 51 which at least substantially sealingly contact inner surface 28 of barrel 22. A front face 52 of plunger head 48 cooperates with inner surface 28 of barrel 22 and an inner face 54 of transition wall 30 to at least partially define a fluid chamber 56 (shown in FIG. 3) of variable volume within main chamber 24. Fluid chamber 56 is in fluid communication with fluid passageway 34. The volume of fluid chamber 56 can be varied by shifting plunger 14 relative to body 12. When plunger 14 is in the retracted position with plunger head 48 being proximate rear portion 20 (shown in FIG. 3), preferably contacting rear portion 20, the volume of fluid chamber 56 is near its maximum. When plunger 14 is in the advanced position with plunger head 48 being proximate front portion 18, preferably contacting front portion 18 (shown in FIG. 4), the volume of fluid chamber 56 is near its minimum, preferably substantially zero.

Shaft 42 of plunger 14 is configured to be extendable through opening 38 and at least partly shiftable within main chamber 24. Shaft 42 presents an outer surface 58 which is generally cylindrical in shape. A recess 60 is defined in outer surface 58 at an attachment location 61 and is configured to facilitate the coupling of barrier 16 to plunger 14.

Attachment location 61 is preferably generally centrally located on shaft 42 between proximal end 46 and distal end 50. The position of attachment location 61 along shaft is perhaps best described in terms of the position of attachment location 61 relative to barrel 22 when plunger 14 is in the advanced position (shown in FIG. 4). If barrel 22 were partitioned longitudinally into three sections of equal length (i.e., a front zone, a middle zone, and a rear zone) attachment location 61 would preferably be located in the middle zone, spaced from both proximal end 46 and distal end 50, when plunger 14 is in the advanced position (shown in FIG. 4).

It is preferred for barrier 16 to be attached to shaft 42 at the specific location described above because it has been unexpectedly discovered that attaching the barrier in this manner optimizes the amount of material required to form barrier 16, and provides optimal functionality of syringe 10. If barrier 16 were coupled to or proximate plunger head 48 (i.e., in the front zone of barrel 22 when plunger 14 is in the advanced position), an excessive amount of barrier material would be required in order to prevent barrier 16 from automatically pulling plunger 14 rearward when plunger 14 is in the advanced position. However, using such an excessive amount of barrier material would cause an undesirably large amount of loose barrier material to gather at or near rear opening 38 when plunger 14 is in the retracted position. Further, if barrier 16 were coupled to or proximate disk 44 (i.e., in the rear zone of barrel 22 when plunger 14 is in the advanced position), an excessive amount of barrier material would be required in order to prevent barrier 16 from automatically pulling plunger 14 forward when plunger 14 is in the retracted position. However, using such an excessive amount of barrier material would cause an undesirably large amount of loose barrier material to gather at or near rear opening 38 when plunger 44 is in the advanced position. By positioning attachment location 61 generally centrally along barrel 22 when plunger 14 is in the advanced position, the risk of automatic advancement or retraction of plunger 14 is minimized and the required amount of barrier material is optimized.

Barrier 16 preferably comprises a highly flexible material which can be deformed when plunger 14 is shifted relative to body 12. More preferably, barrier 16 can be composed of a thin elastomeric material which can be deformed when plunger 14 is shifted relative to body 12 and thereafter returned to substantially its original shape. Still more preferably, barrier 16 is composed of a thin sheet of resilient synthetic material selected from the group consisting of polyurethane, polypropylene, polyethylene, and latex. Most preferably, barrier 16 is composed of a thin latex material.

Barrier 16 is preferably formed of a unitary piece of material which presents two openings. One of the openings is adapted to be coupled to plunger 14, while the other of the openings is adapted to be coupled to body 12. Barrier 16 can be coupled to plunger 14 and body 12 by means known in the art. Preferably, barrier 16 includes elastic sealing rings 62 and 64 proximate each of the openings. Plunger sealing ring 62 is adapted to encircle and be received within recess 60 of plunger 14. Plunger sealing ring 62 exerts a radially inward compressive force on shaft 42 to thereby form a seal between sealing ring 16 and shaft 42. Body sealing ring 64 is adapted to encircle barrel 22 proximate flange 36. Body sealing ring 64 exerts a radially inward compressive force on barrel 22 to thereby form a seal between body sealing ring 64 and barrel 22.

A sterile chamber 66 is cooperatively defined, at least in part, by an inner surface 68 of barrier 66, a rear face 70 of plunger head 48, and an internal portion 72 of outer surface 58 of shaft 42. Further, unless plunger 14 is in the fully retracted position, a portion of inner surface 28 also defines at least a portion of sterile chamber 66. Thus, plunger head 48 separates sterile chamber 66 from fluid chamber 56, with sterile chamber 66 and fluid chamber 56 being on generally opposite sides of plunger head 48.

Although the outer surface of syringe 10 can be contacted with contaminants present in the external environment, sterile chamber 66 is isolated from such contaminants. Barrier 16, therefore, protects any portion of inner surface 28 which can define fluid chamber 56 from contaminants which could otherwise enter through rear opening 38 and contact inner surface 28. Thus, barrier 16 prevents the contamination of an injection fluid contained in fluid chamber 56 with contaminants entering through opening 38.

The materials of construction for the various components of syringe 10 can be any suitable materials known in the art for constructing similar syringes. Preferably, body 12 is made of a relatively translucent synthetic resin material. Preferably, shaft 42 and disk 14 are formed of a relatively rigid synthetic resin material. Preferably, plunger head 48 is composed of a relatively resilient synthetic resin material capable of forming a seal with body 12.

The preferred forms of the invention described above are to be used as illustration only, and should not be utilized in a limiting sense in interpreting the scope of the present invention. Obvious modifications to the exemplary embodiment, as herein above set forth, could be readily made by those skilled in the art without departing from the spirit of the present invention.

The inventor hereby states his intent to rely on the Doctrine of Equivalents to determine and assess the reasonably fair scope of the present invention as pertains to any apparatus not materially departing from but outside the literal scope of the invention as set forth in the following claims.

1. A syringe comprising:
an elongated shaft presenting first and second ends;
a plunger head coupled to the first end; and
a barrier sealingly coupled to the shaft at an attachment location generally centrally positioned between the first and second ends.

2. A syringe as claimed in claim 1,
said barrier and said plunger head cooperating to at least partly define a sterile chamber which is isolated from the environment outside the syringe.

3. A syringe as claimed in claim 2,
said shaft including an internal portion and an external portion,
said internal portion including the first end and disposed in the sterile chamber,
said external portion including the second end at least partly contacting the environment outside the syringe.

4. A syringe as claimed in claim 1,
said barrier comprising an elastomeric material.

5. A syringe as claimed in claim 1,
said barrier comprising latex.

6. A syringe as claimed in claim 1,
said barrier including an elastomeric sealing ring for sealingly coupling the barrier to the shaft.

7. A syringe as claimed in claim 6,
said shaft defining a recess at the attachment location for receiving the elastomeric sealing ring.

8. A syringe as claimed in claim 7,
said shaft presenting a generally cylindrical outer surface.

9. A syringe comprising:
a body including a front portion, a rear portion, and an elongated barrel extending between the front and rear portions;
a plunger head received in the barrel and shiftable relative to the barrel between an advanced position in which the plunger head is located proximate the front portion and a retracted position in which the plunger head is located proximate the rear portion;
an elongated shaft coupled to the plunger head and extending out of the body at the rear portion; and
a barrier sealingly coupled to the body and the shaft, said barrier coupled to the shaft at an attachment location which is positioned generally centrally along the length of the barrel when the plunger head is in the advanced position.

10. A syringe as claimed in claim 9,
said plunger head and said body cooperating to at least partly define a fluid chamber of variable volume.

11. A syringe as claimed in claim 10,
said barrier and said plunger head cooperating to at least partly define a sterile chamber.

12. A syringe as claimed in claim 11,
said sterile chamber and said fluid chamber being positioned on generally opposite sides of the plunger head.

13. A syringe as claimed in claim 11,
said sterile chamber isolated from the environment outside the syringe.

14. A syringe as claimed in claim 11,
said sterile chamber operable to isolate any portion of the body which can define the fluid chamber from contaminants located outside the syringe.

15. A syringe as claimed in claim 9,
said barrier presenting first and second open ends.

16. A syringe as claimed in claim 15,
said first open end including a first elastomeric sealing ring for coupling the barrier to the shaft at the attachment location,
said second open end including a second elastomeric sealing ring for coupling the barrier to the rear portion of the body.

17. A syringe as claimed in claim 16,
said barrier comprising an elastomeric material.

18. A syringe as claimed in claim 9,
said elongated barrel consisting of a front zone, a middle one, and a rear zone, each having equal lengths,
said attachment location being positioned outside the front zone when the plunger head is in the advanced position.

19. A syringe adapted to prevent entry of contaminants from an external environment into an injection fluid disposed in the syringe, said syringe comprising:
a body including a front portion, a rear portion, and an elongated barrel extending between the front and rear portions, said rear portion presenting an opening, said elongated barrel presenting an inner surface; said elongated barrel consisting of a front zone, a middle zone, and a rear zone, each having equal lengths;
a plunger head at least substantially sealingly contacting the inner surface and shiftable relative to the body between an advanced position wherein the plunger head contacts the front portion and a retracted position wherein the plunger head contacts the rear portion, said inner surface and said plunger head cooperating to at least partly define an injection fluid chamber of variable volume;
an elongated shaft coupled to the plunger head and at least partly extending through the opening in the rear portion; and
a barrier sealingly coupled to the rear portion and the elongated shaft, said barrier coupled to the elongated shaft at an attachment location,
said attachment location being positioned in the middle zone of the barrel when the plunger head is in the advanced position.

20. A syringe as claimed in claim 19,
said barrier and said plunger head cooperating to at least partly define a sterile chamber which is isolated from the external environment.

21. A syringe as claimed in claim 19,
said barrier including a first open end and a second open end,
said first open end sealingly coupled to the elongated shaft at the attachment location,
said second open end sealingly coupled to the rear portion.

22. A syringe as claimed in claim 19,
said barrier comprising an elastomeric material.

23. A syringe as claimed in claim 19,
said barrier operable to prevent entry of the contaminants into the body through the opening.

24. A syringe as claimed in claim 19; and
a sterile chamber at least partly defined by the barrier and the plunger head,
said sterile chamber being located on the side of the plunger head generally opposite the fluid chamber.

25. A syringe as claimed in claim 24,
said sterile chamber operable to isolate any portion of the body which can define the fluid chamber from the external environment.

26. A method of assembling a syringe, said method comprising the steps of:
(a)sealingly coupling a flexible barrier to an elongated shaft of a plunger at an attachment location generally centrally positioned on the elongated shaft.

27. A method as claimed in claim 26; and
(b)sealingly coupling the barrier to a body of the syringe.

28. A method as claimed in claim 27,
steps (a) and (b) including deforming an elastomeric sealing ring of the barrier.

29. A method as claimed in claim 28,
step (a) including positioning one of the sealing rings at least partly in a recess formed in the shaft at the attachment location.

30. A method as claimed in claim 29,
step (b) including positioning the other of the sealing rings proximate a flange of the body.
